(19) United States
(12) Patent Application Publication
(10) Pub. No.: US 2023/0223107 A1
(43) Pub. Date: Jul. 13, 2023

(54) METHOD FOR IDENTIFYING SIGNATURES FOR PREDICTING TREATMENT RESPONSE

(71) Applicants: SkylineDx B.V., Rotterdam (NL); UMC Utrecht Holding B.V., Utrecht (NL)

(72) Inventors: Jeroen DE RIDDER, Utrecht (NL); Joske UBELS, Ultrecht (NL)

(21) Appl. No.: 17/995,525

(22) PCT Filed: Apr. 6, 2021
(86) PCT No.: PCT/NL2021/050220

§ 371 Date: Oct. 5, 2022

(30) Foreign Application Priority Data
Apr. 6, 2020 (EP) 20168150.9

(51) Int. Cl. G16B 25/10; G16H 10/20; G16H 20/00; G16H 50/70

(52) U.S. Cl. CPC G16B 25/10; G16H 10/20; G16H 20/00; G16H 50/70

(57) ABSTRACT

The disclosure relates to methods of signatures which can be used in order to classify patients and predict responsiveness to therapy. In particular, the disclosure relates to RAINFOREST (tReAtment benefit prediction using raNdom FOREST), a new method to discover signatures capable of identifying a subgroup of patients more likely to benefit from a specific treatment as compared to another treatment.

### FIELD OF THE INVENTION

The disclosure relates to methods of signatures which can be used in order to classify patients and predict responsiveness to therapy. In particular, the disclosure relates to RAINFOREST (tReAtment benefit prediction using raNdom FOREST), a new method to discover signatures capable of identifying a subgroup of patients more likely to benefit from a specific treatment as compared to another treatment.

### BACKGROUND OF THE INVENTION

Novel drugs are tested for efficacy in phase 3 clinical trials. Despite enormous investments in the development and research prior to the trial, approximately 54% of the phase 3 clinical trials still fail, most often due to a lack of efficacy of the drug tested (Hwang et al. 2016). Trials testing anti-cancer drugs have a higher failure rate than non-cancer drug trial. It was found that trials which adopt a biomarker strategy, i.e. attempt to identify a subset of patients most likely to benefit, have a significantly lower failure rate (Jardim et al. 2017). This is also true for trials evaluating targeted drugs. It is thus clear that even if a clinical trial does not reach its predefined endpoint, there could still be a subset of patients that do see benefit from the drug. Moreover, even if a clinical trial does indicate statistically significant benefit, this benefit may in fact be quite modest and driven by a subset of patients that have a larger benefit from the drug. For this reason, the benefit for all patients may be insufficient to warrant prescription of a drug with very serious side effects. Therefore, it is important to establish which subset of patients benefit more than the population as a whole and develop tools that can predict such treatment benefit at the moment of diagnosis.

It has become clear that the genetic background of both tumor and patient can influence drug response and several germline variants have been linked to the effectiveness of a number of drugs (anti-cancer and other). SNP panels enabling the use of these variants for personalized medicine are under active development (van der Wouden et al. 2019). For instance, for several chemotherapies, its sensitivity or toxicity has been linked to specific single nucleotide polymorphisms (SNPs) (Panczyk 2014; Sullivan et al. 2014; Yin et al. 2012). Despite this initial progress, for many drugs there is no clear relationship between response and a single variant or other simple molecular biomarker and more complex machine learning models are needed. Another important consideration is that most efforts that aim to find biomarkers to predict treatment benefit focus on predicting sensitivity to one specific treatment, i.e. distinguish between poor and good responders within one homogeneous treatment group. However, owing to recent progress in drug development for most cancers there are different treatment options available. A clinically more relevant question is thus whether an individual patient will benefit more from one treatment than another. Therefore, there is a great clinical need for methods to identify markers that can identify subgroups of patients which are likely to benefit from treatment as this may i) rescue failed clinical trials and/or ii) identify subgroups of patients which benefit more than the population as a whole.

### SUMMARY OF THE INVENTION

The disclosure provides the following preferred embodiments. However, the invention is not limited to these embodiments.

The disclosure provides a machine-implemented method for identifying a signature that identifies subgroups of individuals which have a better survival outcome with a treatment of interest, relative to an alternative therapy, said method comprising
providing data from a group of individuals, said data comprising for each individual (i) a plurality of genetic marker data and/or expression data for a plurality of genes, (ii) treatment arm data, and (iii) survival data;
calculating a survival difference (SurvDiff) for each genetic marker and/or for each gene;
using a random forest model to train multiple tree classifiers, wherein each individual decision tree is trained on a different subset of the genetic markers and/or genes and wherein for each node in the tree a calculation of the SurvDiff is used as splitting criterion;

whereby the trained random forest model identifies a signature that can distinguish subgroups of individuals which have a better survival outcome with the therapy of interest, relative to an alternative treatment.

Preferably, each genetic marker or gene expression is coded as a ternary value, preferably wherein the ternary value is 0, 1 or 2.

Preferably the survival difference (SurvDiff) for each individual genetic marker and/or gene is calculated for >0 and >1.

Preferably the genetic marker data and/or expression data is germline data or tumor cell genetic data, preferably wherein the data is germline data.

Preferably the genetic markers are SNPs (single nucleotide polymorphisms).

Preferably for each individual the survival data is known or imputed.

Preferably the calculation of the survival difference score is based on the survival data, treatment arm data and the number of individuals included.

Preferably the survival difference score represents the absolute difference between the survival data in the left node of the split and the right node of the split.

Preferably the survival difference score is calculated by

$$SurvDiff = \vert \frac{-}{\sqrt{\frac{{σ ( {survA}_{L} )}^{2}}{{n}_{{A}_{L}}} + \frac{σ ( {survB}_{L} )}{{n}_{{B}_{L}}}}} - \frac{-}{\sqrt{\frac{{σ ( {survA}_{R} )}^{2}}{{n}_{{A}_{R}}} + \frac{σ ( {survB}_{R} )}{{n}_{{B}_{R}}}}} \vert$$

Preferably wherein a hazard ratio is calculated, whereby a hazard ratio below 1 indicates benefit from receiving the treatment.

Preferably the data was obtained from clinical trials, preferably wherein individuals are randomly assigned to one or more treatment arms.

Preferably the data from individuals does not have classification labels.

Preferably the data is obtained from individuals having cancer.

Preferably the data is obtained from individuals having colorectal cancer, preferably metastatic colorectal cancer.

### BRIEF DESCRIPTION OF THE DRAWINGS

FIG. 1. An overview of one embodiment of the RAINFOREST algorithm The survival curves show examples of what a class ‘benefit’ and ‘no benefit’ should look like. We train 10,000 of these individual decision trees to form the RAINFOREST model, which is validated on ⅓ of the data that acts as test data and was not used in training of the model.

FIG. 2. a. Scatterplot of the T-test statistic and Cox regression coefficient per SNP. We perform this analysis once using the reference allele to define class ‘benefit’ and once using the alternative allele. b. Kaplan Meier of the CAIRO2 survival data used, showing no survival benefit for the patients who received cetuximab. c. The HR found in class ‘benefit’ when using different threshold on the posterior probability to define benefit. The red dashed line shows the HR between treatments found in the dataset as a whole, without any classification. d. Kaplan Meier of the classification in class ‘benefit’ and ‘no benefit’ using the posterior probability threshold associated with the lowest Cox regression p-value in class ‘benefit’.

FIG. 3. a. Manhattan plot showing the number of times individual SNPs were used in a decision tree across all three cross validation folds. b. Venn diagram showing the overlap in SNPs used in the three models for the three different cross validation folds. c. Barplot showing the 20 SNPs with the greatest influence on validation HR when the data is shuffled. Error bars indicate standard deviation. The SNPs indicated in red text are in LD>0.9 with each other and all lie in the same region of chromosome 5. SNPs in black are not in high LD with any other SNP in the plot.

FIG. 4 a. The OOB error found for the survival based levels when using different values for mtry. b. Kaplan Meier of the classification in class ‘benefit’ and ‘no benefit’, using the threshold that defines the class ‘benefit’ with the lowest Cox regression p-value.

### DETAILED DESCRIPTION OF THE DISCLOSED EMBODIMENTS

One of the advantages of the methods described herein is the ability to define treatment benefit as having a better survival outcome on the treatment of interest than an alternative treatment. We aim to predict treatment benefit based on genetic and/or expression data from patients. To apply machine learning for this purpose there are two main challenges to be addressed. First of all, traditional class labels required for training machine learning models are not available. It is not possible to know how a patient would have responded to a treatment they did not receive, and therefore one cannot know a priori whether a patient benefited or not (and thus label them as such). More specifically, a patient responding well to a certain treatment could have had an even better response on an alternative treatment. Conversely, a poor response does not necessarily mean the patient did not see any benefit from the treatment. This lack of training labels renders most regular machine learning approaches unsuitable. Secondly, genome wide germline variation datasets are very high dimensional, often including 100- to 1000-fold more features (e.g., genetic markers or gene expression data) than samples (patients). As a result, machine learning models have a high risk of overtraining (Szymczak et al. 2009). As is clear to a skilled person, machine learning refers to computer algorithms in particular those that automatically improve through experience and/or the use of data.

One class of models, which has shown great promise in preventing overtraining in such situations, are Random Forests (RFs). Outside the cancer field, RFs have successfully been used to predict drug response using germline variation data (Athreya et al. 2019; Cosgun, Limdi, and Duarte 2011). RFs are ensemble classifiers combining multiple decision trees. RFs are explicitly designed to prevent overtraining by using only a subset of the available training samples and randomly sampling a subset of the features at each split. Since the algorithm only has access to part of the dataset at a time, it is less likely to overtrain on the dataset as a whole, while predictive performance remains high due to the fact that many trees are combined in an ensemble. For instance, RFs have been successfully employed to predict optimal warfarin dose using genome wide germline variation data and shown to outperform alternative models (Cosgun, Limdi, and Duarte 2011).

In some embodiments, the methods address the clinically relevant question: which treatment out of several available treatments is the best choice for the patient? To achieve this, a machine learning method is provided that can derive a benefit prediction model from data gathered in a clinical trial in which patients were randomly assigned to different treatment arms, e.g, to one of two different treatment arms. To circumvent the need for classification labels and deal with the extremely high dimensionality of the data, an alternative formulation of the traditional RF classifier is provided (referred to herein as RAINFOREST (tReAtment benefit prediction using raNdom FOREST)).

In one aspect, the disclosure provides a machine-implemented method for identifying a signature that identifies subgroups of individuals which have a better survival outcome with a treatment of interest, relative to an alternative therapy. The method comprises providing data from a group of individuals, said data comprising for each individual (i) a plurality of genetic marker data and/or expression data for a plurality of genes, (ii) treatment arm data, and (iii) survival data. The method further comprises calculating a survival difference (SurvDiff) for each genetic marker and/or for each gene and using a random forest model to train multiple tree classifiers, wherein each individual decision tree is trained on a different subset of the genetic markers and/or genes and wherein for each node in the tree a calculation of the SurvDiff is used as splitting criterion. As is clear to a skilled person, “machine-implemented” refers to computer-implemented.

The disclosed method identifies a signature that can distinguish subgroups of individuals which have a better survival outcome with the therapy of interest relative to an alternative treatment. As used herein, the term signature refers to genetic markers or gene expression which can distinguish subgroups of individuals which have a better survival outcome with the therapy of interest, relative to an alternative treatment.

The signature identified by the method disclosed herein is a predictive signature; or rather it provides information about a therapeutic intervention. The signature identified by the disclosed methods can be used to identify subgroups of individuals that can benefit from a treatment of interest, in particular when this treatment of interest is compared to an alternative therapy. The disclosed methods may be used to identify a signature that identifies subgroups of individuals which have a better survival outcome with a treatment of interest.

The term “better survival outcome” refers to the time until an event may occurs and, for example, may refer to the likelihood that patient survival will increase as a result of the therapy of interest. As is clear to a skilled person, the term better survival outcome refers to a probability and not that 100% of all patients that are predicted to respond to a treatment may actually respond. A skilled person is able to determine when a greater treatment benefit (or difference in time to event) is significant. Preferably, the significance is p>0.05. In some embodiments, the time to event is more than 10%, more than 20%, or more than 50% longer for the greater treatment benefit.

One of the advantages of applying the methods disclosed herein to predict response is that it allows for optimizing a treatment regime. Based on the data of an individual, this individual can be classified as responder or non-responder to the treatment of interest as compared to an alternative treatment. A responder is expected to benefit from the treatment of interest. The non-responders are not expected to benefit from the treatment of interest as compared to the alternative treatment. Individuals that are predicted to respond to a particular treatment may be subsequently administered such treatment. Conversely, individuals predicted not to respond to a particular treatment may be administered with an alternative treatment. This can result in a decrease in unnecessary treatments.

Accordingly, a method is also provided comprising classifying an individual as having a better survival outcome with a treatment of interest relative to an alternative therapy based on the presence of a signature identified according to the disclosed methods.

The machine-implemented method comprises providing data from a group of individuals. A skilled person can determine a group size that will provide enough information to perform the method. Preferably, data from at least 50 individuals is used, more preferably data from at least 100 individuals is used. Preferably, the data is obtained from individuals having the same or closely related disease. Preferably, the data is obtained from individuals having cancer. In a preferred embodiment the data is obtained from individuals having colorectal cancer, preferably metastatic colorectal cancer. Preferably, the therapy is a cancer therapy, in particular a therapy for treating colorectal cancer. Preferably, the therapy is an antibody. Preferably, the therapy is an epidermal growth factor receptor (EGFR) inhibitor, such as cetuximab.

The data may be obtained from available studies or may be obtained specifically for training of the model. In preferred embodiments the data is obtained from clinical trials, preferably wherein individuals are randomly assigned to one or more treatment arms. Clinical trials are experiments or observations done in clinical research. These prospective studies are designed to answer specific questions about biomedical interventions, for example new treatments. Clinical trials generate data on the safety and efficacy of potential new treatments. Novel drugs are tested for efficacy in phase 3 clinical trials. Some clinical trials include data, such as genetic marker data and/or expression data of genes. The data from these clinical trials may be used for the machine-implemented method as disclosed herein.

The data from a group of individuals comprises treatment arm data. As is well-known to a skilled person, for many diseases it is not possible to have a placebo arm. This is especially true for life-threatening diseases. With the disclosed method, a new treatment may be compared to, e.g., the standard of care. In these cases, the disclosed methods are useful for identifying signatures that can predict an increase in responsiveness to the therapy of interest over the standard of care, i.e., an alternative treatment. In some embodiments, the methods are for classifying a subgroup of individuals, in particular, for classifying as benefiting from a therapy of interest as compared to an alternative treatment.

As will be appreciated by a skilled person, the methods disclosed herein are not limited to a disorder or to a particular treatment. In an exemplary embodiment, the signature can be used to identify a subgroup of individuals that can benefit from addition of cetuximab for the treatment of colorectal cancers. For this specific example the treatment of interest is capecitabine, oxaliplatin, bevacizumab and cetuximab, while the alternative treatment is capecitabine, oxaliplatin, bevacizumab. The signature can help to identify the individuals that are likely to benefit from the addition of cetuximab.

The data from a group of individuals also comprises data on time until event, preferably survival data. Response to treatment can be measured by any number of time to events/endpoints including survival time, time-to-disease-progression (TTP), Overall Survival (OS), or Progression Free Survival (PFS). Preferably, the time to event is Survival time. In addition, the time to event can also include the time until a tumor reaches a particular size or the time until a particular symptom appears.

In some embodiments, for each individual the time to event data (e.g., survival) is known or imputed. In some instances, the time to event data for all individuals may be known. For example, there may be clinical trials where the time to event is survival and all patients have had an event. In other cases, for some patients an event may not yet have occurred. In such instances, an event time is imputed based on all patients for whom an event was observed as reference. The examples disclosed herein describe an exemplary embodiment of imputing time to event data.

The data from a group of individuals also comprises genetic marker data and/or expression data for a plurality of genes. In some embodiments, the data comprises data for at least 10, 50, 100, or 1000 different genetic markers. In some embodiments, the data comprises expression data for at least 10, 50, 100, or 1000 different genes.

In some embodiments, the data is gene expression data. As used herein, a gene refers to a sequence of nucleotides in DNA or RNA that encodes either for a protein or a non-coding RNA (e.g., transfer RNAs, ribosomal RNA, microRNAs, etc). Preferably, the gene expression data refers to the expression of a protein encoding gene. There are many published sources of gene expression data, e.g., those obtained from published clinical trials. Gene expression data may also be determined as part of the methods disclosed herein.

Gene expression data refers to the level of nucleic acid or protein expression. In some embodiments, nucleic acid or protein is purified from the sample and expression is measured by nucleic acid or protein expression analysis. Determining the level of expression includes the expression of nucleic acid, preferably mRNA, or the expression of protein.

The level of protein expression can be determined by any method known in the art including ELISAs, immunocytochemistry, flow cytometry, Western blotting, proteomic, and mass spectrometry.

Expression data also refers to the level of nucleic acid. Preferably, the nucleic acid is RNA, such as mRNA or pre-mRNA. As is understood by a skilled person, the level of RNA expression determined may be detected directly or it may be determined indirectly, for example, by first generating cDNA and/or by amplifying the RNA/cDNA. In a preferred embodiment, the expression data is RNA (preferably mRNA or poly-A RNA) expression data.

The level of expression need not be an absolute value but rather a normalized expression value or a relative value. Preferably, the level of expression refers to a “normalized” level of expression. Normalization is particularly useful when expression is determined based on microarray data. Normalization allows for correction for variation within microarrays and across samples so that data from different chips can be simultaneously analyzed. The robust multi-array analysis (RMA) algorithm may be used to pre-process probe set data into gene expression levels for all samples. (Irizarry R A, et al., Biostatistics (2003) and Irizarry R A, et al., Nucleic Acids Res. (2003)). In addition, Affymetrix's default preprocessing algorithm (MAS 5.0), may also be employed. Additional methods of normalizing expression data are described in US20060136145.

In some embodiments, this expression data corresponds to the probes used for detection or the corresponding genes they refer to. Suitable probes include those commercially available on microarrays, such as Affymetix™ chips. It is well within the purview of a skilled person to develop additional probes for determining expression. The level of nucleic acid expression may be determined by any method known in the art including RT-PCR, quantitative PCR, Northern blotting, gene sequencing, in particular RNA sequencing, and gene expression profiling techniques. In some embodiments, the level of expression is determined using a microarray. In some embodiments, the level of expression is determined using RNA sequencing.

In some embodiments, the data is genetic marker data. As used herein, “genetic markers” refers to specific DNA sequences with a known location on a chromosome or specific RNA sequences encoded by DNA sequences with a known location of a chromosome. Suitable genetic markers include not only mutations but also genetic polymorphisms (i.e., alternative sequences at a locus that occur among individuals or populations of individuals). Suitable genetic markers include SNPs, indels, structural variations, inversions, rearrangements, duplications, satellite repeats (e.g., macro-satellites, mini-satellites, and micro-satellites), copy number variations, etc. Suitable genetic markers can be identified by, for example, comparing genetic sequences between the individuals in the study (e.g., those that received a treatment) or by comparing genetic sequences between individuals in the study with a reference human genome sequence (e.g., GRCh37, GRCh38). Any sequences which vary among individuals or populations of individuals are suitable. Various databases are also available which describe genetic markers, see, e.g., the world wide web at ncbi.nlm.nih.gov/snp/ which contains human single nucleotide variations, microsatellites, and small-scale insertions and deletions along with publication, population frequency, molecular consequence, and genomic and RefSeq mapping.

In a preferred embodiment, the genetic markers are SNPs. The term “single nucleotide polymorphism” or “SNP” as used herein refers to a genetic variation in the DNA sequence that occurs at a single nucleotide position. The density of SNPs in the human genome is estimated to be approximately 1 per 1,000 base pairs.

Methods for determining genetic markers are known in the art. While such markers can be detected using RNA, DNA is generally preferred. Such methods include restriction fragment length polymorphism, mass spectrometry, and hybridization analysis. Preferably, genetic markers are determined by DNA sequencing. Such sequencing may include whole or partial genome sequencing or whole or partial exome sequencing. Suitable methods include high-throughput and next generation sequencing (see, e.g., Teama “DNA Polymorphisms: DNA-Based Molecular Markers and Their Application in Medicine”, Genetic Diversity and Disease Susceptibility 2018).

Typical samples for collecting genetic marker data and/or expression data for a plurality of genes are tissues and bodily fluids, such blood, serum, plasma, urine, cerebrospinal fluid, and saliva. In some embodiments, the genetic marker data and/or expression data is germline data. In some embodiments, the genetic marker data and/or expression data is specific for a tumor. In some embodiments, this data may relate to somatic mutations or somatic mutations that effect expression. Methods for identifying and obtaining tumor samples are well known to a skilled person. Many clinical trials include data collected from tumor samples and/or collected from non-tumorous samples (i.e., germline data).

In a preferred embodiment, each genetic marker is coded as a ternary value, preferably wherein the ternary value is 0, 1, or 2. For example, the absence of a particular marker may be coded as 0, the presence of the marker on one chromosome may be coded as 1, and the presence of the marker on both chromosomes may be coded as 2. A skilled person will further appreciate that the data can also be coded into, e.g., four different groups, such as 0, 1, 2, or 3.

In some embodiments the data is coded as a ternary value, preferably wherein the ternary value is 0, 1 or 2. In some embodiments the data is gene expression data. Such data can be made ternary, for example by using two thresholds (e.g. 25^{th} percentile and 75^{th} percentile), leading to the coding of below both thresholds (would be coded as, e.g., 0), in between the thresholds (would be coded as, e.g., 1), and above both thresholds (would be coded as, e.g., 2). A skilled person recognizes that alternative methods exist for converting continuous gene expression to a ternary alternative. A skilled person will further appreciate that the gene expression data can also be coded into, e.g., four different groups, such as 0, 1, 2, or 3.

In some embodiments, the data is genetic marker data. This data can also be coded in a ternary fashion. For example, if both chromosome copies at a certain position in the cells of interest (e.g. tumor cells) have the same result as germline DNA (e.g. from blood), that would be coded as 0. Should one of the two chromosome copies have a variation (e.g., insertion, deletion, or nucleotide substitution), it will be coded as 1. Should both copies of the chromosome have the variation, it will be coded as 2. A skilled person recognizes that alternative methods exist for converting sequence data to a ternary value.

In an exemplary embodiment, the genetic marker is a SNP. In some embodiments, the SNP is a bi-allele polymorphism and an individual may be homozygous or heterozygous for an allele at each SNP location. For example, at a particular position in the human genome a particular nucleotide (e.g., T) may appear in most individuals. In a minority of individuals, this position is occupied by a different nucleotide (e.g. A). By way of example only, an individual being homozygous for a T at the position indicated above would be coded as 0. An individual being heterozygous, having on one allele a T and the other an A, would be coded as 1. An individual being homozygous for the SNP (in this example an A), would be coded as 2. A skilled person recognizes that the method works equally well if the values are assigned in reverse, e.g., with an individual being homozygous for a T at the position indicated coded as 2 and an individual being homozygous for a A at the position indicated coded as 0. Similar coding may be performed with other genetic markers.

The methods disclosed herein utilize an adaptation of the random forest model to predict treatment benefit from patient genetic marker profiles and gene expression data. Instead of using the Gini impurity, which is traditionally used to decide on the best possible split in a decision tree, the methods use survival difference (SurvDiff). SurvDiff captures the survival difference between the treatment arms and does not rely on an a priori specification of class labels. Instead, the SurvDiff measure enables training decision trees by providing a split criterion, which results in a ‘benefit’ and ‘no benefit’ branch in the tree. Preferably, the calculation of the survival difference score is based on the survival data, treatment arm data and the number of individuals included.

A decision tree is a type of data structure used to store data about the best features for the model accumulated during a training phase so that it may be used to make predictions about examples previously unseen by the decision tree. Multiple decision trees can be used as part of an ensemble of decision trees (referred to as a random forest) trained for a particular application domain in order to achieve generalization (that is being able to make good predictions about examples which are unlike those used to train the forest). This generalization is moreover achieved, by randomly sampling a part of the data one decision tree is trained on. Since every tree has access to different features and a slightly different part of the samples, the random forest is less specific for the training data set and thus more likely to perform well on new data. A decision tree has a first node called a root node, a plurality of split nodes and a plurality of leaf nodes. Leaf nodes are nodes without a child node. During training the structure of the tree (the number of nodes and how they are connected) is learned as well as split functions to be used at each of the split nodes. In addition, data is accumulated at the leaf nodes during training

Data from an individual can be pushed through each of the decision trees of a random forest. At each split node a decision is made based on the data from a genetic marker or gene expression.

By way of example only, at a split node, the data points proceed to the next level of the tree down the chosen branch. During the training of the model, the ternary value of the data are learnt for use at the split nodes. Data are accumulated at the leaf nodes. In some embodiments every tree is restricted to a depth of two. This restriction helps to prevent overtraining of the model and leads to a tree with a maximum number of four leaves and means that every tree uses at most three data point (i.e., genetic markers or gene expression). Preferably, every tree uses at most three genetic markers or the expression from three different genes.

In some embodiments, the node is only split further when it contains a sufficient number of individuals, for example, at least 50 individuals. This is to prevent the random forest to be biased towards choosing non-informative data points; for example, non-informative SNPs with a high minor allele frequency over informative SNPs with a lower allele frequency. This bias is not very pronounced in the beginning of a tree, but can dramatically influence the data selection lower in the tree, when the sample sizes are smaller.

When a certain value for an allele or gene (i.e. gene expression) is more common for individuals who benefit from the treatment of interest compared to an alternative treatment, this allele or gene has predictive value for treatment benefit. These alleles or genes for which a difference is seen between the responders and non-responders will result in a higher SurvDiff score as compared to a random allele/gene with less predictive value. The best predictive allele/gene is the one resulting in the maximum value of SurvDiff.

Preferably, the SurvDiff is calculated based on the ternary value of the allele/gene, the survival data for the individuals, and the treatment each individual has received. The SurvDiff scores for the alleles/gene can be used to build decision trees.

In some embodiments, SurvDiff is the absolute difference between the survival score in the left node of the split compared to the right node of the split. In an exemplary embodiment, there is a survival score calculated for each node of the split, left and right. A higher absolute difference between the left and right node after the split indicates predictive value of the allele/gene. This score for each arm is calculated by the difference of the mean survival data for the individuals having one allele versus the other allele or having a high or low expression of a particular gene. In some embodiments the survival difference score represents the absolute difference between the survival data in the left node of the split and the right node of the split.

The methods comprise calculating a survival difference (SurvDiff) for each SNP and/or for each gene. In some embodiments the survival difference (SurvDiff) for each individual genetic marker and/or gene is calculated for >0 and >1. For example, in case of SNP alleles the ternary score can be 0, 1 or 2 for each individual. In an exemplary embodiment, the score for each arm is calculated twice. First, the difference of the mean survival data for the individuals having allele 0 (ternary value) versus the mean survival data for the individuals having allele 1 or 2 (ternary value; >0) is calculated. Second, the difference of the mean survival data for the individuals having allele 0 or 2 (ternary value) versus the mean survival data for the individuals having allele 2 (ternary value; >1) is calculated.

In one embodiment the survival difference score is calculated by:

$$SurvDiff = \vert \frac{-}{\sqrt{\frac{{σ ( {survA}_{L} )}^{2}}{{n}_{{A}_{L}}} + \frac{σ ( {survB}_{L} )}{{n}_{{B}_{L}}}}} - \frac{-}{\sqrt{\frac{{σ ( {survA}_{R} )}^{2}}{{n}_{{A}_{R}}} + \frac{σ ( {survB}_{R} )}{{n}_{{B}_{R}}}}} \vert$$

In this formula [symbol] and [symbol] are the mean survival data for treatment arm A and B in the left node of a split, respectively. Similarly, [symbol] and [symbol] are the equivalent in the right node of a split. Moreover, n_{A} and n_{B} denote the number of samples included in the node in treatment arm A and B, respectively. For example, if the provided data comprises SNP allele data, each SNP under consideration is tested at two thresholds (SNP value >0 or >1) to define the left and right node. In the above example, SurvDiff thus corresponds to calculating the absolute difference between an “unpaired” or “independent samples” t-test, such as the Welch's T-test, found in the left and right node. The best SNP in this example is the one resulting in the maximum value of SurvDiff.

In one embodiment the method is used to calculate a hazard ratio. A hazard ratio is the ratio of the hazard rates corresponding to the conditions described by two levels of an explanatory variable. A hazard ratio below 1 indicates benefit from receiving the treatment. The hazard ratio associated with a treatment provides an estimate of the hazard of experiencing progression of disease relative to the hazard when another treatment would be given. In the absence of training labels that can be used to calculate accuracy, the hazard ratio is used as performance measure when validating the RAINFOREST model in cross validation.

In preferred embodiments the data from individuals does not have classification labels. For many data sets traditional classification labels which are required for training machine learning models are not available. It is unknown how an individual would have responded to a treatment they did not receive, and therefore we cannot know a priori whether an individual benefited or not (and thus label them as such). For example, an individual responding well to a certain treatment could have had an even better response on an alternative treatment. Conversely, a poor response does not necessarily mean the individual did not see any benefit from the treatment. The lack of classification labels renders most regular machine learning approaches unsuitable.

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

All patent and literature references cited in the present specification are hereby incorporated by reference in their entirety.

The invention is further explained in the following examples. These examples do not limit the scope of the invention, but merely serve to clarify the invention.

### EXAMPLES

When phase III clinical drug trials fail their end-point, enormous resources are wasted. Moreover, even if a clinical trial demonstrates a significant benefit, the observed effects are often rather small and may not outweigh the side effects of the drug. Therefore, there is a great clinical need for methods to identify markers that can identify subgroups of patients which are likely to benefit from treatment as this may i) rescue failed clinical trials and/or ii) identify subgroups of patients which benefit more than the population as a whole. When single genetic biomarkers cannot be found, machine learning approaches that find multivariate signatures are required. In the context of SNP profiles and gene expression data this is extremely challenging owing to the high dimensionality of the data. Here we introduce RAINFOREST (tReAtment benefit prediction using raNdom FOREST), an adaptation of the random forest that can predict treatment benefit from patient genetic marker profiles and gene expression data obtained in a clinical trial setting.

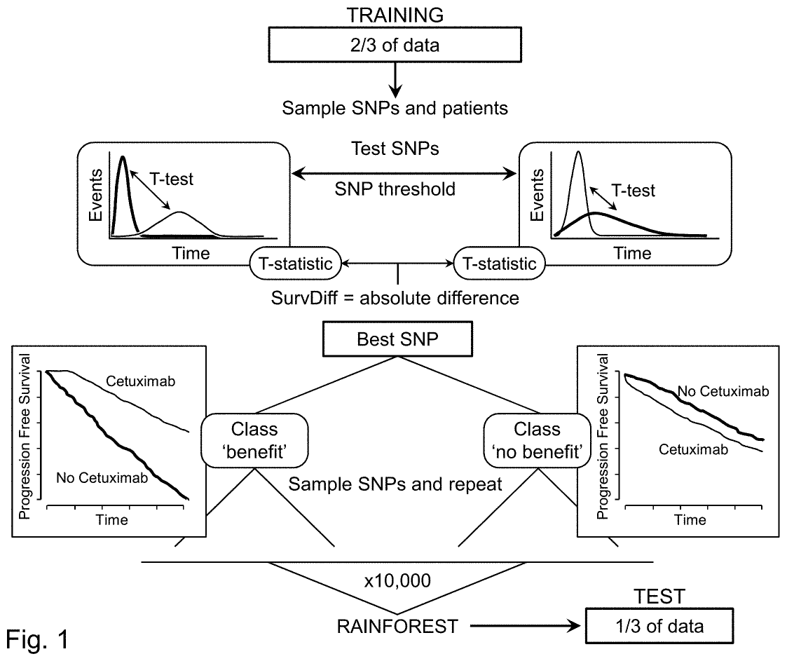

In some embodiments of RAINFOREST, the Gini impurity, which is traditionally used to decide on the best possible split in a decision tree, is replaced by the SurvDiff measure. SurvDiff captures the survival difference between the treatment arms and does not rely on an a priori specification of class labels. Instead, the SurvDiff measure enables training decision trees by providing a split criterion, which results in a ‘benefit’ and ‘no benefit’ branch in the tree. An overview of a preferred embodiment of RAINFOREST and the SurvDiff measure is provided in FIG. 1.

We apply RAINFOREST to the CAIRO2-trial, a randomized phase III clinical trial designed to test whether patients with metastatic colorectal cancer benefit from addition of the EGFR inhibitor cetuximab to standard first-line treatment. This trial showed that the addition of cetuximab to a regimen of chemotherapy and bevacizumab results in a significantly shorter progression free survival (Tol et al. 2009). However, it is known that cetuximab response varies widely between patients. Previously, several somatic mutations in the tumor that influence cetuximab response have been identified (Salvatore et al. 2010; Khan et al. 2017). Moreover, in the context of the CAIRO2 trial a germline SNP was identified with the potential capability to predict treatment benefit (Pander et al. 2015), although this variant was not validated.

In the following examples we demonstrate the capability of RAINFOREST on the CAIRO2 trial. We show that RAINFOREST can identify a subset of patients with significant benefit from cetuximab and that this approach outperforms both univariate analysis and a random forest trained on predefined labels. While the CAIRO2 trial concluded there was no benefit, surprisingly RAINFOREST is able to identify a subgroup comprising 27.7% of the patients that significantly benefit from treatment with a hazard ratio of 0.69 (p=0.04) in favor of cetuximab.

This method is not specific to colorectal cancer or the specific CAIRO2 trial and can be applied, for example, to other clinical trial data and provide a more personalized approach to cancer treatment. In particular in cases for drugs where there is no clear link between a single variant and treatment benefit.

Methods

1.1 Overview of RAINFOREST

A random forest model is an ensemble classifier consisting of individual decision trees trained on different subsets of the training data. More specifically, each tree in the forest only has access to a subset of the samples (sampled with replacement) and for each split in the tree a random subset of the features is sampled.

The optimization of each tree, i.e. choosing the optimal split for a node in the tree, is most often achieved by minimizing the Gini impurity. The Gini impurity is a measure of the probability that a sample would be incorrectly labeled in this split and is 0 when a node contains only samples with the same label. Problematically, in the context of predicting treatment benefit no predefined training labels are available, as we cannot know if a patient survived longer (or shorter) from treatment than on standard of care or some other treatment. We can therefore not use the Gini impurity for RF construction.

Treatment effect is most often determined through a Cox proportional hazards model (see next section for more details), based on which a hazard ratio (HR) is calculated. The HR associated with a treatment provides an estimate of the hazard of experiencing progression of disease relative to the hazard when another treatment would be given. A HR below 1 indicates benefit from receiving the treatment. In the absence of training labels that can be used to calculate accuracy, we use the HR as performance measure when validating the RAINFOREST model in cross validation. Problematically, estimating a Cox model is too computationally expensive to be used in a splitting criterion when training thousands of decision trees. We therefore propose RAINFOREST, a random forest approach in which we introduce a novel splitting criterion that can be optimized to directly predict treatment benefit. For each sample, RAINFOREST can use treatment arm data, survival data and patient data (e.g., genetic marker or gene expression data). Each decision tree should define a class ‘benefit’ and ‘no benefit’ which maximizes the difference between treatment effect. We define this difference through the splitting criterion SurvDiff:

$$SurvDiff = \vert \frac{-}{\sqrt{\frac{{σ ( {survA}_{L} )}^{2}}{{n}_{{A}_{L}}} + \frac{σ ( {survB}_{L} )}{{n}_{{B}_{L}}}}} - \frac{-}{\sqrt{\frac{{σ ( {survA}_{R} )}^{2}}{{n}_{{A}_{R}}} + \frac{σ ( {survB}_{R} )}{{n}_{{B}_{R}}}}} \vert \qquad ( 1 )$$

where [symbol] and [symbol] are the mean survival data for treatment arm A and B in the left node of the split, respectively. Similarly, [symbol] and [symbol] are the equivalent in the right node. Moreover, n_{A} and n_{B} denote the number of samples included in the node in treatment arm A and B, respectively. For each SNP under consideration we test two thresholds (SNP value >0 or >1) to define the left and right node. SurvDif f thus corresponds to calculating the absolute difference between the Welch's T-test statistics found in the left and right node. The best SNP is the one resulting in the maximum value of SurvDiff.

Using this criterion we trained 10,000 decision trees. We further prevented overtraining by restricting every tree to a depth of two. This restricts the tree to a maximum number of four leaves (nodes without a child node) and means every tree uses at most three SNPs. When building a tree using SNP data, the RF can be biased towards choosing non-informative SNPs with a high minor allele frequency over informative SNPs with a lower minor allele frequency (Boulesteix et al. 2012). This bias is not very pronounced in the beginning of a tree, but can dramatically influence SNP selection lower in the tree, when smaller sample sizes are present. We therefore also only split a node further when it contains at least 50 patients. These restrictions also reduce computational cost. An overview of the construction of the RAINFOREST model is given in FIG. 1.

1.2 Survival Analysis and Event Imputation

Survival data is right censored, which means that all patients who did not experience progression of disease by the end of follow-up are censored, i.e. no event is recorded. Cox models can handle censored data by maximizing the partial log likelihood over coefficient β through:

[symbol]

(β)=Σ_{i:C}_{ᵢ}₌₁Xᵢ*β−logΣ_{j:Y}_{ⱼ}_{≥Y}_{ᵢ}θⱼ (2)

where θ_j=exp(X_j*β) and X represents the explanatory variable, i.e. the treatment arm in this situation. When estimating the likelihood of an event occurring for subject i at a certain time t the θj_is summed for every subject j that has not yet experienced an event at t. In this way censored patients can be included and used for optimization up to the time of censoring, instead of being excluded from the dataset all together.

The HR is defined as the exponent of 6.

The SurvDiff measure does not rely on Cox models. Instead, RAINFOREST deals with the censoring problem by imputation. More specifically, for all censored patients an event time is imputed based on all patients for whom an event was observed as reference. To achieve this, a Weibull distribution is fitted to all uncensored patients through maximum likelihood estimation. The Weibull distribution can be used to adequately parametrize a survival distribution and can also—akin Cox regression—model proportional hazards (Carroll 2003). The cumulative distribution function of a Weibull distribution is described by

F(x; k, λ)=1−e^{-(x/λ}^{ₖ}) (3)

where x is the time to event, k is a shape parameter and λ is the scale parameter. In our dataset we find the maximum likelihood is reached with a value of 11.91 for λ and 1.65 for k. Importantly, we find very similar parameters for the distribution when we perform a maximum likelihood estimation for each treatment arm separately, justifying an estimation over the whole dataset. This is in line with the observation in the original trial that there is no significant survival difference between the two treatment arms. For each censored patient we now sample an event time greater than the time of censoring from the estimated Weibull distribution.

1.3 Data

In this work the survival and genome wide genotype data from patients enrolled in the CAIRO2 trial are used, which included patients in 79 Dutch centers to test the addition of cetuximab for the treatment of metastatic colorectal cancers. The data generation and processing has been previously described in detail (Pander et al. 2015). Briefly, we use survival data and germline DNA of 553 patients who received treatment regimen CAPDX-B (capecitabine, oxaliplatin and bevacizumab) with cetuximab (n=279) or without cetuximab (n=274).

DNA was isolated from peripheral leukocytes and genome wide genotyping was performed with Illumina beadchip arrays. Of all measured variants 647,550 passed all quality checks and we performed no imputation of additional variants. We also exclude SNPs with a minor allele frequency <5% and SNPs with any missing data, after which 257,008 SNPs remain. Each SNP is coded as 0, 1, or 2, corresponding to the number of copies of the alternative allele. We use progression free survival (PFS) as the end point in all analyses.

1.4 Univariate SNP Analysis

To evaluate the ability of individual SNPs to predict cetuximab benefit, we computed two Cox proportional hazard models per SNP. First, we computed an additive model which contains the SNP and treatment arm as separate variables. The second model also includes an interaction term between the SNP and treatment arm (i.e. treatment arm*SNP). For a SNP that influences treatment benefit, this second model should provide a better fit. We tested whether there is a significant difference in model fit using a likelihood ratio test. We ranked SNPs on most significant contribution of the interaction term to the model, as measured by the likelihood ratio test p-value. With the best SNPs we define a benefit score by:

$$benefitScore = {\sum }_{i = 1}^{n} {X}_{i} {β}_{i} \qquad ( 4 )$$

Where X is the alternative allele count for a certain SNP i and β the Cox regression coefficient associated with the interaction term. We performed forward feature selection to determine the best SNP combination by ranking the SNPs on p-value and adding the top 250 in order. The SNP combination resulting in the lowest HR in class ‘benefit’ is chosen. We validated this model in a three fold cross validation

1.5 Random Forest Using Survival-Derived Labels

We compared the performance of RAINFOREST to the results obtained by a regular RF trained on the survival labels directly (which, as discussed previously, is not necessarily the best measure for treatment benefit). To obtain a labeled dataset, required for training a regular RF, we defined the class ‘benefit’ as the patients with the 25% best progression free survival from the cetuximab arm combined with the patients with the 25% worst progression free survival from the other arm. The other 75% of patients comprise class ‘no benefit’. With these labels we defined a class benefit that has a significantly better survival on cetuximab than the rest of the population, satisfying our definition of treatment benefit.

1.6 Cross Validation Fold Construction

To evaluate the performance of univariate SNP selection, the regular RF and the RAINFOREST models, we employed 3-fold cross validation. To ensure the results are directly comparable, we used the same folds for all analyses. To obtain a fair estimation of the performance, it is important that the different folds are stratified, i.e. contain a similar and representative part of the whole dataset. Here we cannot balance the folds using training labels, as these are not available. To ensure the cross validation folds are representative, we therefore balanced on treatment arm. Furthermore, we require that the HR found between the treatment arms does not differ more than 0.05 between all three folds.

1.7 Optimization of Mtry Parameter

RFs often use an out-of-bag (OOB) error to optimize model parameters. Since in an RF model each tree samples a different subset of the patients, each training sample is not used in a number of trees. The OOB error is determined by classifying each training sample, using only the trees in which a particular sample was not included. However, the OOB error can severely underestimate performance when random sampling is performed from unbalanced classes (Mitchell 2011). As we do not know the labels here, representative sampling is impossible. Using random sampling we indeed see that the OOB performance, defined as the HR between treatment arms in class ‘benefit’, is close to random (HR class ‘benefit’ in OOB sample is 1.45 (95% CI 0.94-2.26, p=0.10)).

As we cannot obtain a realistic estimation of the performance from the OOB sample in RAINFOREST, we cannot optimize the mtry parameter which defines how many features are sampled at every split. However, previous work suggests that the best mtry is linked to dataset dimensionality (Goldstein et al. 2010). The RF trained on survival labels uses the same features as RAINFOREST. In training this RF we tried several settings for mtry (√p, 2√p, 0.1p and 0.2p). For training RAINFOREST we used the same mtry setting as in the best performing RF trained on survival based labels (√p) and trained 10,000 trees.

Results

2.1 T-Test in SurvDiff Criterion Captures Survival Difference

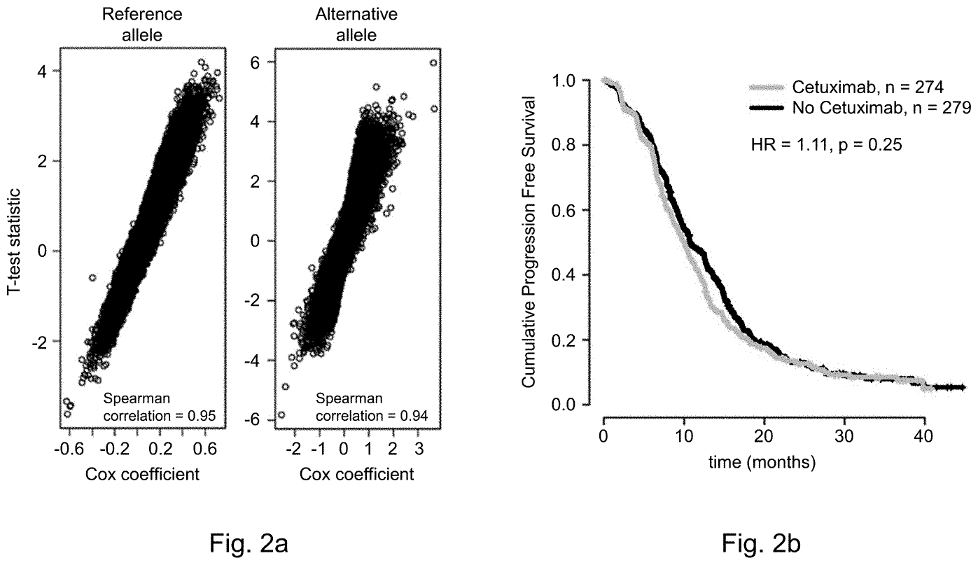

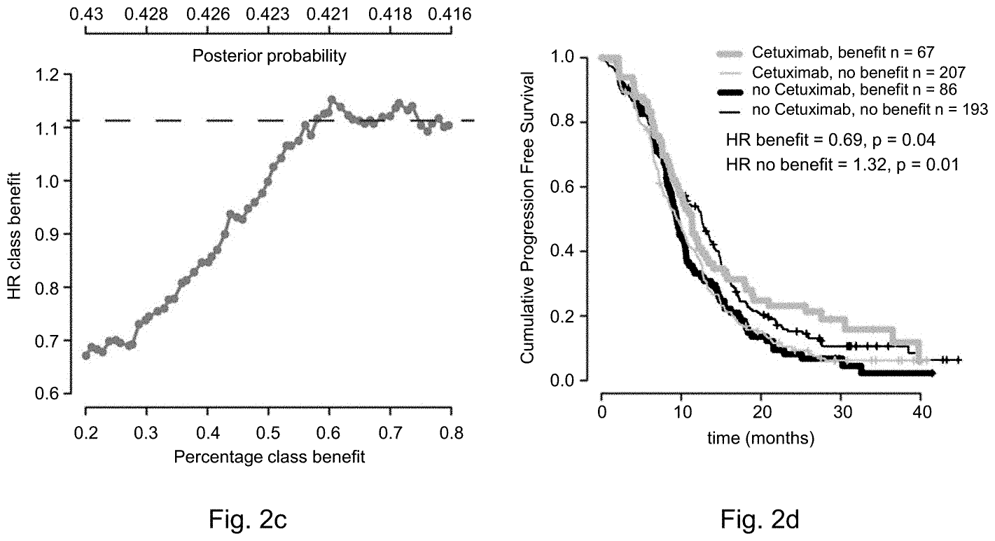

We first assessed whether the T-test on the imputed survival data, which is used in the SurvDiff splitting criterion, captures the same signal as Cox regression would capture, to ensure this is a suitable measure to use during training of the RAINFOREST model. For each SNP we performed a T-test for both the reference and alternative allele, contrasting the difference in imputed survival between the two treatment arms. We compared the resulting T-test statistic to the equivalent Cox regression β (FIG. 2a). We find these measures to be highly correlated for both the reference allele (Spearman correlation coefficient=0.95, p<2*10⁻¹⁶) and the alternative allele (Spearman correlation coefficient=0.94, p<2*10⁻¹⁶). Importantly, this approach reduces compute time by one order of magnitude (34.41 minutes for the Cox regression computation versus 1.89 for the T-test on a single core). Thus, the T-test approach captures a similar signal as a full survival analysis while keeping it computationally feasible to train a model with thousands of trees.

2.2 RAINFOREST can Identify Patients Benefiting from Cetuximab

We next trained RAINFOREST to predict cetuximab benefit on the CAIRO2 trial data and validate its performance in a three-fold cross validation. FIG. 2b shows the survival curves in the dataset as a whole, without any classification. Here we find an HR of 1.11 (95% CI 0.93-1.33, p=0.25) for cetuximab treatment. FIG. 2c shows the different HRs found in class ‘benefit’ when using different operating points of the classifier (i.e. different thresholds on the number of trees classifying a sample as ‘benefit’). This curves indicates a direct relationship between the operating point and the HR found in class ‘benefit’—we find a lower HR when the threshold is set higher. As no sample has a posterior probability higher than 0.5, we have to adjust this threshold. This also results in a smaller class ‘benefit’ and there is thus a trade off between the size of class ‘benefit’ and the HR found. FIG. 2d shows the Kaplan Meier plot when the classification threshold that results in the lowest p-value in class ‘benefit’ is used. We show the combined results across the three cross validation folds, i.e. the predictions for each patient is based on the two folds in which this patient was not present. In class ‘benefit’ (n=153) we find a significant HR of 0.69 (95% CI 0.49-0.98, p=0.04) whereas in class ‘no benefit’ (n=400) an HR of 1.32 (95% CI 1.07-1.62, p=0.01) is found. This performance is relatively stable in all cross validation folds; we find an HR of 0.66 (0.33-1.03, p=0.23) in class ‘benefit’ in Fold 1, an HR of 0.72 (0.40-1.31, p=0.28) in Fold 2, and an HR of 0.61 (0.44-1.09, p=0.10) in Fold 3. While the original trial concluded addition of cetuximab to the standard regimen has no benefit, this result shows RAINFOREST can successfully identify a subset of patients, comprising 27.7% of the population, that do benefit from cetuximab.

2.3 Known and New SNPs are Identified in Frequently Chosen SNPs

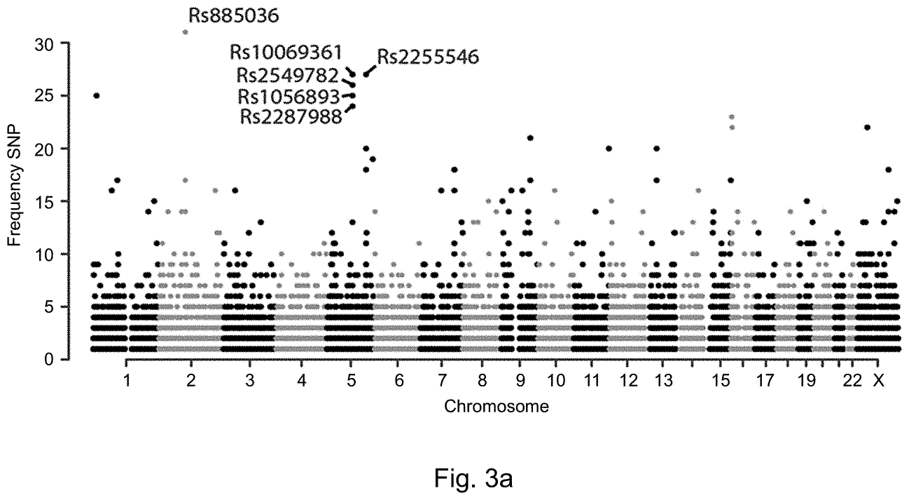

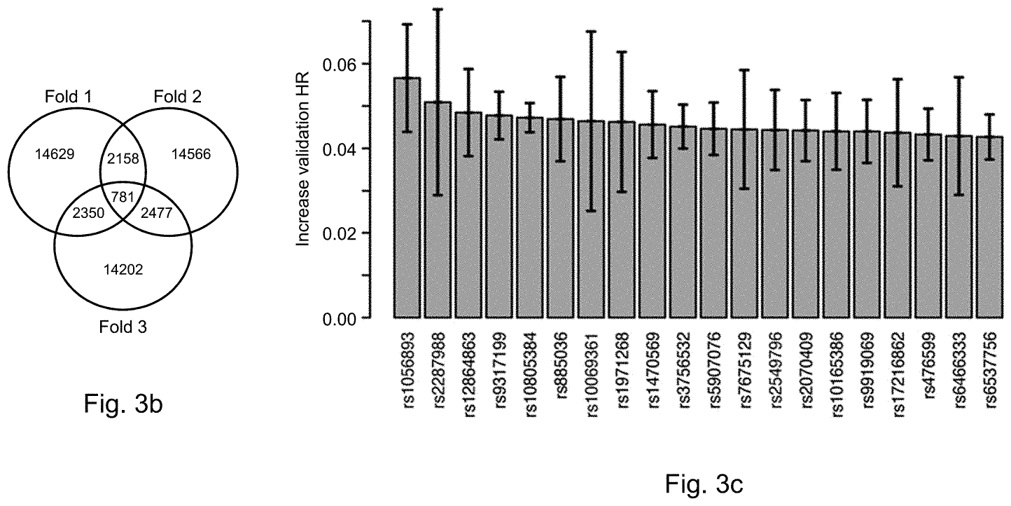

Over the three cross validation folds in total 51,154 unique SNPs are used (19,918, 19,982, and 19,810 in the models validated on Fold 1, 2 and 3 respectively). FIG. 3b shows the number of SNPs overlapping between the three different models. We obtained an empirical p-value for this overlap by randomly sampling 10,000 trees for each fold and computing the overlap. We find the overlap of 781 SNPs between the three folds to be significant (p<1*10⁻⁴). We also trained a RAINFOREST model using shuffled treatment labels with the same cross validation folds. With shuffled labels the association between genomic data and treatment specific outcome is removed and these models can indeed not predict benefit in hold-out data (HR class benefit=0.95, 95% CI 0.64-1.41, p=0.8). Between the models trained on shuffled labels only 356 SNPs overlap, which is similar to mean overlap found in random sampling (mean overlap=344.7). The overlap found in the RAINFOREST model is thus clearly non-random.

FIG. 3a shows the number of times each individual SNP is selected across the three cross validation folds. Interestingly, the SNP selected most often, rs885036, has been reported before to predict cetuximab benefit in a univariate analysis of the CAIRO2 trial (Pander et al. 2015). This shows that when univariate signals are present in the data, RAINFOREST will also capture these. In addition to rs885036, we also find a cluster of frequent SNPs on chromosome 5 which have not been reported before. Four of these variants (rs2549782, rs2287988, rs1056893 and rs2255546) are intronic variants within the ERAP1 gene. A fifth SNP (rs10069361) is annotated to LNPEP, a paralog of ERAP1. These SNPs are in high linkage disequilibrium (coefficient of linkage disequilibrium >0.9), where linkage disequilibrium is defined as the squared Pearson correlation coefficient. Both ERAP1 and LNPEP code for aminopeptidases. ERAP1 plays an important role in cleaving proteins into peptides that can be presented by MHC class 1 proteins to immune cells (Falk and Rotzschke 2002). Cetuximab is a monoclonal antibody and it has been shown that activation of the adaptive of the immune system and presence of cytotoxic T-cells are essential for its antitumor effect (Holubec et al. 2016; Yang et al. 2013). A potential explanation of these observations is that these SNPs represent genetic variation in the T-cell response that influence cetuximab response.

For all 781 SNPs that are present in all three models we also assessed feature importance by shuffling the genotype of the individual SNP and predicting the class labels on the validation again. This eliminates the association between the genetic data and treatment effect, so we can estimate the importance of each SNP. Without exception, shuffling SNPs increases the HR, which means the model performs worse. FIG. 3c shows the difference in HR for the 20 SNPs with the largest effect. Note that since many SNPs are only present in a few trees (i.e. the most frequent SNP is only present 31 times), the effect of shuffling is limited. We thus also do not see large changes in validation HR. Despite this limitation, 4 out of 5 SNPs from the chromosome 5 cluster as well as rs885036 are present in the top 20, strengthening their putative role in predicting cetuximab benefit.

2.4 Univariate SNP Selection Does Not Validate in Cross Validation

We compared the performance of RAINFOREST to the univariate selection of SNPs (see Methods). This analysis reveals no SNPs that are significant at a multiple testing corrected p-value less than 0.05. We performed forward feature selection by ranking the SNPs on likelihood ratio test p-value to find the optimal SNP combination. With this approach, the models for fold 1, 2 and 3 contain 101, 197 and 190 SNPs respectively . In line with the earlier univariate study (Pander et al, 2015), Rs8885036 (the most frequently selected SNP in the RAINFOREST model) is selected in all three folds. With the exception of one other SNP (rs10165386) no other SNPs overlap. Moreover, the model does not result in a significant HR, as we find an HR of 1.00 (95% CI=0.70-1.44, p=1) in class ‘benefit’ (n=138) and an HR of 1.15 (95% CI 0.93-1.41, p=0.19) in class ‘no benefit’ (n=415). Univariate selection of the SNPs thus does not lead to a model that validates on unseen patient data.

3.5 Random Forest on Survival Based Labels Does Not Validate

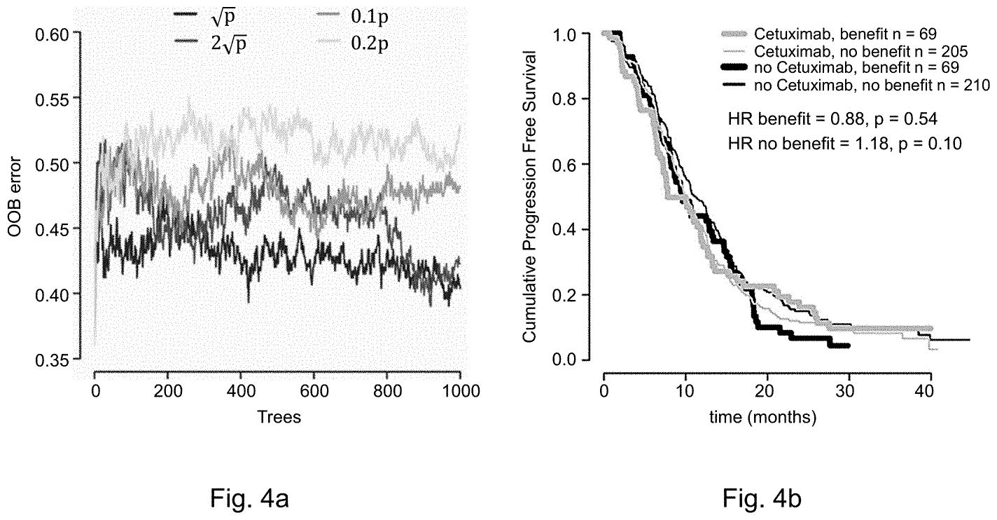

We also trained a classical random forest model on the benefit labels derived from the survival data (see Methods). The cross validation is performed using the same folds as in the univariate and RAINFOREST analysis. Since we do have training labels in this case, mtry can be optimized using the OOB error. The default setting often used is the square root of all features available, but it has been suggested that in high dimensional datasets a higher mtry leads to a better performance (Goldstein et al. 2010). We therefore tried several values for mtry and evaluate the OOB error. FIG. 4a shows that the default \sqrt{p}, where p is the total number of features, leads to the lowest error (FIG. 4a).

Using the optimal model we find that no patients are classified into the ‘benefit’ class when using majority vote, despite the fact that both classes are sampled equally in the training data.

We therefore classify a sample with where more than 30% of trees indicate benefit as benefiting, as this leads to a class benefit of approximately 25%. Using these settings we train a random forest with 10,000 trees and validate it on the test set. In the test set we set a threshold on the posterior probability that results in the lowest p-value in class ‘benefit’. We then find an HR of 0.88 (95% CI 0.59-1.32, p=0.54) in class ‘benefit’ (n=138) and an HR of 1.18 (95% CI 0.97-1.44, p=0.10) in class ‘no benefit’ (n=415). The Kaplan Meier curve is shown in FIG. 4b. While the RF can identify a class ‘benefit’ with an HR below 1, this is not statistically significant at p<0.05. Similar results are obtained when defining benefit as the top 50% and bottom 50% of the treatment arms (HR benefit=0.97, 95% CI 0.70-1.36, p=0.88) or when restricting the RF to a depth of two (HR benefit=0.92, 95% CI 0.50-1.67, p=0.77). We conclude that predefined benefit labels based on survival outcome are not suitable as training labels for training a RF classifier for treatment benefit.

Discussion

We here demonstrate RAINFOREST, a new approach to predict treatment benefit from patient data. The RAINFOREST model successfully identifies a subset of patients that benefits from cetuximab treatment in the CAIRO2 trial. It outperforms univariate analysis and traditional random forest models. We demonstrate its performance through cross validation, as the best estimate of the performance on independent validation data. In this model we have only considered the influence of germline variation on cetuximab benefit. Several tumor characteristics, like KRAS and BRAF mutation status and molecular subtype, have also been shown to correlate with cetuximab response (Salvatore et al., 2010, Trinh et al, 2017). Thus, RAINFOREST is also expected to identify tumor specific markers.

The CAIRO2 trial represents a good test case for RAINFOREST as previous univariate analysis has shown a relation between germline variation and treatment specific survival. Reassuringly, we identify rs8885036, the variant identified previously, among the most frequently used SNPs in the RAINFOREST model. Importantly, RAINFOREST identifies a number of previously unknown SNPs, which are not found with a univariate approach, that suggest a role for genetic variation in the immune response in determining cetuximab benefit.

The authors of the CAIRO2 trial concluded that there was a slight detrimental effect of the addition of cetuximab to the CAPDX-B treatment regimen. This is a clear example for how RAINFOREST can be applied, as roughly half of all phase 3 clinical trials fail to reach their predefined endpoints and most fail due to insufficient efficacy of the drug (Hwang et al. 2016). As a result, these drugs do not enter the clinic, while it is very possible that a subset of the patient population experiences benefit. RAINFOREST can identify patients that do benefit from drugs which failed to show significant benefit in the patient population as a whole, and thus play an important role in leveraging valuable patient data and find an application for drugs that otherwise would not be introduced to the clinic.

### REFERENCES

Athreya, A. P., et al. 2019. “Pharmacogenomics-Driven Prediction of Antidepressant Treatment Outcomes: A Machine-Learning Approach With Multi-trial Replication.” Clinical Pharmacology & Therapeutics. https://doi.org/10.1002/cpt.1482.
Boulesteix, A. et al. 2012. “Random Forest Gini Importance Favours SNPs with Large Minor Allele Frequency: Impact, Sources and Recommendations.” Briefings in Bioinformatics 13 (3): 292-304.
Carroll, K. J. 2003. “On the Use and Utility of the Weibull Model in the Analysis of Survival Data.” Controlled Clinical Trials. https://doi.org/10.1016/s0197-2456(03)00072-2.
Cosgun, E et al. 2011. “High-Dimensional Pharmacogenetic Prediction of a Continuous Trait Using Machine Learning Techniques with Application to Warfarin Dose Prediction in African Americans.” Bioinformatics 27 (10): 1384-89.
Falk, K, and Rotzschke, 0.2002. “The Final Cut: How ERAP1 Trims MHC Ligands to Size.” Nature Immunology. https://doi.org/10.1038/ni1202-1121.
Goldstein, B. A., et al. 2010. “An Application of Random Forests to a Genome-Wide Association Dataset: Methodological Considerations & New Findings.” BMC Genetics 11 (June): 49.
Holubec, L et al. 2016. “The Role of Cetuximab in the Induction of Anticancer Immune Response in Colorectal Cancer Treatment.” Anticancer Research. https://doi.org/10.21873/anticanres.10985.
Hwang, T. J. et al. 2016. “Failure of Investigational Drugs in Late-Stage Clinical Development and Publication of Trial Results.” JAMA Internal Medicine 176 (12): 1826-33.
Jardim, D. L. et al. 2017. “Factors Associated with Failure of Oncology Drugs in Late-Stage Clinical Development: A Systematic Review.” Cancer Treatment Reviews 52 (January): 12-21.
Khan, S. A. et al. 2017. “EGFR Gene Amplification and KRAS Mutation Predict Response to Combination Targeted Therapy in Metastatic Colorectal Cancer.” Pathology Oncology Research: POR 23 (3): 673-77.
Mitchell, Matthew W. 2011. “Bias of the Random Forest Out-of-Bag (OOB) Error for Certain Input Parameters.” Open Journal of Statistics. https://doi.org/10.4236/ojs.2011.13024.
Panczyk, Mariusz. 2014. “Pharmacogenetics Research on Chemotherapy Resistance in Colorectal Cancer over the Last 20 Years.” World Journal of Gastroenterology: WJG 20 (29): 9775-9827.
Pander, J. et al. 2015. “Genome Wide Association Study for Predictors of Progression Free Survival in Patients on Capecitabine, Oxaliplatin, Bevacizumab and Cetuximab in First-Line Therapy of Metastatic Colorectal Cancer.” PloS One 10 (7): e0131091.
Salvatore, M. Di et al. 2010. “KRAS and BRAF Mutational Status and PTEN, cMET, and IGF1R Expression as Predictive Markers of Response to Cetuximab plus Chemotherapy in Metastatic Colorectal Cancer (mCRC).” Journal of Clinical Oncology. https://doi.org/10.1200/jco.2010.28.15_suppl.e14065.
Sullivan, I. et al.. 2014. “Pharmacogenetics of the DNA Repair Pathways in Advanced Non-Small Cell Lung Cancer Patients Treated with Platinum-Based Chemotherapy.” Cancer Letters 353 (2): 160-66.
Szymczak, S. et al 2009. “Machine Learning in Genome-Wide Association Studies.” Genetic Epidemiology. https://doi.org/10.1002/gepi.20473.
Tol, J., et al. (2009). Chemotherapy, Bevacizumab, and Cetuximab in Metastatic Colorectal Cancer. New England Journal of Medicine, 360(6), 563-572. https://doi.org/10.1056/NEJMoa0808268
Trinh, A. et al. (2017) Practical and Robust Identification of Molecular Subtypes in Colorectal Cancer by Immunohistochemistry. Clinical Cancer Research 23 (2), 387 -398
Wouden, C. H. et al. 2019. “Development of the PGx-Passport: A Panel of Actionable Germline Genetic Variants for Pre-Emptive Pharmacogenetic Testing.” Clinical Pharmacology and Therapeutics 106 (4): 866-73.
Yang, X et al. 2013. “Cetuximab-Mediated Tumor Regression Depends on Innate and Adaptive Immune Responses.” Molecular Therapy: The Journal of the American Society of Gene Therapy 21 (1): 91-100.
Yin, J et al.2012. “Meta-Analysis on Pharmacogenetics of Platinum-Based Chemotherapy in Non Small Cell Lung Cancer (NSCLC) Patients.” PloS One 7 (6): e38150.

1. A machine-implemented method for identifying a signature that identifies subgroups of individuals which have a better survival outcome with a treatment of interest, relative to an alternative therapy, said method comprising
providing data from a group of individuals, said data comprising for each individual (i) a plurality of genetic marker data and/or expression data for a plurality of genes, (ii) treatment arm data, and (iii) survival data;
calculating a survival difference (SurvDiff) for each genetic marker and/or for each gene;
using a random forest model to train multiple tree classifiers, wherein each individual decision tree is trained on a different subset of the genetic markers and/or genes and wherein for each node in the tree a calculation of the SurvDiff is used as splitting criterion;
whereby the trained random forest model identifies a signature that can distinguish subgroups of individuals which have a better survival outcome with the therapy of interest, relative to an alternative treatment.

2. Machine-implemented method according to claim 1, wherein each genetic marker or gene expression is coded as a ternary value.

3. Machine-implemented method according to claim 2, wherein the survival difference (SurvDiff) for each individual genetic marker and/or gene is calculated for >0 and >1.

4. Machine-implemented method according to claim 1, wherein the genetic marker data and/or expression data is germline data or tumor cell genetic data.

5. Machine-implemented method according to claim 1, wherein the genetic markers are SNPs (single nucleotide polymorphisms).

6. Machine-implemented method according to claim 1, wherein for each individual the survival data is known or imputed.

7. Machine-implemented method according to claim 1, wherein the calculation of the survival difference score is based on the survival data, treatment arm data and the number of individuals included.

8. Machine-implemented method according to claim 1, wherein the survival difference score represents the absolute difference between the survival data in the left node of the split and the right node of the split.

9. Machine-implemented method according to claim 1, wherein the survival difference score is calculated by
$$SurvDiff = \vert \frac{-}{\sqrt{\frac{{σ ( {survA}_{L} )}^{2}}{{n}_{{A}_{L}}} + \frac{σ ( {survB}_{L} )}{{n}_{{B}_{L}}}}} - \frac{-}{\sqrt{\frac{{σ ( {survA}_{R} )}^{2}}{{n}_{{A}_{R}}} + \frac{σ ( {survB}_{R} )}{{n}_{{B}_{R}}}}} \vert$$

10. Machine-implemented method according to claim 1, wherein a hazard ratio is calculated, whereby a hazard ratio below 1 indicates benefit from receiving the treatment.

11. Machine-implemented method according to claim 1, wherein the data was obtained from clinical trials.

12. Machine-implemented method according to claim 11, wherein the data from individuals does not have classification labels.

13. Machine-implemented method according to claim 1, wherein the data is obtained from individuals having cancer.

14. Machine-implemented method according to claim 13, wherein the data is obtained from individuals having colorectal cancer.

15. Machine-implemented method according to claim 2, wherein the ternary value is 0, 1 or 2.

16. Machine-implemented method according to claim 11, wherein individuals are randomly assigned to one or more treatment arms.
